(19) United States
(12) Patent Application Publication
(10) Pub. No.: US 2024/0366642 A1
(43) Pub. Date: Nov. 7, 2024

(54) COMPRESSED TABLETS COMPRISING HMO

(71) Applicant: DSM IP Assets B.V., Heerlen (NL)

(72) Inventors: Zhenbo MA, Kaiseraugst (CH); Conroy Clive SALMON, Kaiseraugst (CH); Paulo Henrique SANTOS, Kaiseraugst (CH)

(21) Appl. No.: 18/255,673

(22) PCT Filed: Nov. 26, 2021
(86) PCT No.: PCT/EP2021/083079

§ 371 Date: Jun. 2, 2023

Related U.S. Application Data
(60) Provisional application No. 63/121,407, filed on Dec. 4, 2020.

(30) Foreign Application Priority Data
Jan. 12, 2021 (CH) 00018/21

(51) Int. Cl. A61K 31/702; A61K 9/20; A61K 31/4045; A61K 45/06

(52) U.S. Cl. CPC A61K 31/702; A61K 9/2018; A61K 9/2054; A61K 9/2095; A61K 31/4045; A61K 45/06

(57) ABSTRACT

The present invention relates to compressed (robust) tablets comprising a specific amount of at least one human milk oligosaccharide (HMO).

The present invention relates to compressed (robust) tablets comprising a specific amount of at least one human milk oligosaccharide (HMO).

Formation and stability of a robust tablet relies on the use of binding agents in the tablet formulation. Such tablets can be chewable tablets, or regular swallowable tablets, or ODT (orally disintegrating tablets), or any other types of tablets. The use of such tablets is very wide spread.

Many binding agents such as PVP (polyvinylpyrrolidone), and diluents with binding capacities such as sorbitol, mannitol, and MCC (microcrystalline cellulose) have been widely used in dietary supplements and pharmaceutical robust tablet products. However, the use of those binding agents can often be limited for various reasons. For example, sorbitol has laxative effect and can cause gastrointestinal symptoms; MCC is water insoluble and can cause grittiness for chewable tablets and ODT; although PVP is a good binding agent, it is more often used in wet granulation and can cause lot-to-lot variation when applied as a dry binding agent for direct compressible tablets.

Therefore, there is a need for alternative binding agent having good binding properties but not having the disadvantages of the usual binding agents as described above.

Surprisingly, the use of at least one HMO in a robust tablet leads to very robust tablets and the amount of usual binding agents can be reduced.

The tablet hardness is an important quality factor to ensure tablets to remain intact during coating, tumbling, and transportation. In addition, the use of binding agent(s) can help to reduce power consumption because lower compression force is required.

It was found out, that surprisingly the use of at least one HMOs the robustness/hardness of tablets is improved significantly.

The use of HMO can reduce the use of other binding agents such as sorbitol while achieving much more robust tablets. In other words, the use of HMO can also help to lower compression force and power consumption accordingly.

Additionally, the HMOs have also demonstrated various health benefits at different usage rates.

Therefore, the present invention relates to a compressed tablet (CT) comprising
(i) at least one binding agent
(ii) at least 1 wt-%, based on the total weight of the compressed tablet, of at least one HMO, and
(iii) optionally at least one further active ingredient (such as vitamins, nutritional lipids, minerals, carotenoids, nutraceuticals, glycosaminoglycan or its active members, amino acids or combination thereof that are useful in human or animal nutrition, and any other type of dietary ingredient. (Preferably vitamins, minerals, glycosaminoglycan or its active members),
   wherein the compressed tablet has a hardness of at least 2 kp.

1 kp is 9.80665 Newton (N).

Binding agents in the context of the present invention are
Saccharides and their derivatives, such as disaccharides (i.e. sucrose, lactose); polysaccharides and their derivatives (i.e. starches, cellulose or modified cellulose (i.e. microcrystalline cellulose) and cellulose ethers such as hydroxypropyl cellulose (HPC)); sugar alcohols such as xylitol, sorbitol or mannitol;
Proteins (such as gelatin);
Synthetic polymers (such as polyvinylpyrrolidone (PVP), polyethylene glycol (PEG));
Mineral type of binders such as Calcium Carbonate and DiCalcium Phosphate.

Therefore, the present invention relates to a compressed tablet (CT1), which is compressed tablet (CT), wherein the at least binding agent is chosen from the group consisting of saccharides and their derivatives, proteins, synthetic polymers, and mineral type of binders.

Therefore, the present invention relates to a compressed tablet (CT1′), which is compressed tablet (CT) or (CT1), wherein the at least binding agent is chosen from the group consisting of disaccharides (i.e. sucrose, lactose), polysaccharides and their derivatives (i.e. starches, cellulose or modified cellulose (i.e. microcrystalline cellulose and cellulose ethers such as hydroxypropyl cellulose (HPC)); sugar alcohols such as xylitol, sorbitol or mannitol), gelatin, polyvinylpyrrolidone (PVP) and polyethylene glycol (PEG)); mineral type of binders such as Calcium Carbonate and DiCalcium Phosphate.

The amount of the at least one binding agent can vary. A usual (and preferred) range of the at least one binder is 0.5 to 98 wt-%, based on the total weight of the compressed tablet.

More preferred is a range of 20 to 80 wt-%, based on the total weight of the compressed tablet.

Therefore, the present invention relates to a compressed tablet (CT2), which is compressed tablet (CT) or (CT1′), wherein the amount of the at least one binding agent is 0.5 to 98 wt-%, based on the total weight of the compressed tablet.

More preferred is a range of 20 to 80 wt-%, based on the total weight of the compressed tablet.

Therefore, the present invention relates to a compressed tablet (CT2′), which is compressed tablet (CT) or (CT1′), wherein the amount of the at least one binding agent is 20 to 80 wt-%, based on the total weight of the compressed tablet.

An essential ingredient of the compressed tablet according to the present invention is one or more HMO.

Human milk oligosaccharides (HMOs) are a family of structurally diverse unconjugated glycans that are highly abundant in and unique to human milk. Originally, HMOs were proposed to be prebiotic “bifidus factors,” or human milk glycans found to promote growth in Bifidobacterial species of the gut and found uniquely in the stool of breast fed infants compared to formula fed infants.

HMOs are composed of the five monosaccharides glucose (Glc), galactose (Gal), N-acetylglucosamine (GlcNAc), fucose (Fuc) and sialic acid (Sia), with N-acetylneuraminic acid (Neu5Ac) as the predominant if not only form of Sia. More than two hundred different HMOs have been identified so far. The most important ones are 2′-fucosyllactose (2′ FL), lacto-N-neotetraose (LNnT), 3-fucosyllactose (3FL), difucosyllactose (DFL), Lacto-N-fucopentaose I (LNFP I), 3′Sialyllactose Sodium Salt (3′SL), 6′Sialyllactose Sodium Salt (6′SL), and Lacto-N-Tetraose (LNT).

HMOs can be isolated from breast milk or they can be produced chemically or biochemically. HMOs are available commercially from a variety of producers.

For the purpose of the present invention the source of the HMO is not essential. It is clear that HMOs from different sources can be used.

Several studies have reported the health benefits of HMOs, which include modulation of the intestinal microbiota, anti-adhesive effect against pathogens, modulation of the intestinal epithelial cell response, and development of the immune system.

Therefore, HMOs have very positive effect when consumed by humans and/or animals. In the embodiment of the present invention the HMO does not only serve as a binding agent having a positive effect on the property of the compressed tablet but also has a very positive effect when consumed by humans and/or animals.

Therefore the present invention relates to a compressed tablet (CT3), which is the compressed tablet (CT), (CT1), (CT1′), (CT2) or (CT2′), wherein the HMO are chosen from the group consisting of 2′-fucosyllactose (2′ FL), lacto-N-neotetraose (LNnT), 3-fucosyllactose (3FL), difucosyl-lactose (DFL), Lacto-N-fucopentaose I (LNFP I), 3′Sialyllactose Sodium Salt (3′SL), 6′Sialyllactose Sodium Salt (6′SL), and Lacto-N-Tetraose (LNT).

Therefore the present invention relates to compressed tablet (CT3′), which is the compressed tablet (CT), (CT1), (CT1′), (CT2) or (CT2′), wherein the HMO are chosen from the group consisting of 2′-fucosyllactose (2′ FL), lacto-N-neotetraose (LNnT), 3-fucosyllactose (3FL), difucosyl-lactose (DFL), Lacto-N-fucopentaose | (LNFP I), and Lacto-N-Tetraose (LNT).

Therefore the present invention relates to compressed tablet (CT3″), which is the compressed tablet (CT), (CT1), (CT1′), (CT2) or (CT2′), wherein the HMO are chosen from the group consisting of 2′-fucosyllactose (2′ FL) and lacto-N-neotetraose (LNnT).

Therefore the present invention relates to compressed tablet (CT3″), which is the compressed tablet (CT), (CT1), (CT1′), (CT2) or (CT2′), wherein the HMO is lacto-N-neotetraose (LNnT).

In the compressed tablet according to the present invention, the amount of the at least one HMO is 1-60 wt-%, based on the total weight of the compressed tablet. (Preferably 2-55 wt-%, more preferably 3-40 wt-%, especially preferred 5-25 wt-%, based on the total weight of the compressed tablet).

It is clear that the wt-% in a compressed tablet always adds up to 100.

Therefore the present invention relates to a compressed tablet (CT4), which is the compressed tablet (CT), (CT1), (CT1′), (CT2), (CT2′), (CT3), (CT3′), (CT3″) or (CT3′″), wherein the content of the at least one HMO is 1-60 wt-%, based on the total weight of the compressed tablet.

Therefore the present invention relates to a compressed tablet (CT4′), which is the compressed tablet (CT), (CT1), (CT1′), (CT2), (CT2′), (CT3), (CT3′), (CT3″) or (CT3′″), wherein the content of the at least one HMO is 2-55 wt-%, based on the total weight of the compressed tablet.

Therefore the present invention relates to a compressed tablet (CT4″), which is the compressed tablet (CT), (CT1), (CT1′), (CT2), (CT2′), (CT3), (CT3′), (CT3″) or (CT3′″), wherein the content of the at least one HMO is 3-40 wt-%, based on the total weight of the compressed tablet.

Therefore the present invention relates to a compressed tablet (CT4″), which is the compressed tablet (CT), (CT1), (CT1′), (CT2), (CT2′), (CT3), (CT3′), (CT3″) or (CT3′″), wherein the content of the at least one HMO is 5-25 wt-%, based on the total weight of the compressed tablet.

The compressed tablets according to the present invention can also comprise further active ingredients (next to HMOs). Such ingredient are vitamins, nutritional lipids, carotenoids, minerals, nutraceuticals, glycosaminoglycan or its active members, amino acids or combination thereof that are useful in human or animal nutrition, and any other type of dietary ingredients.

These ingredients can be present in an amount of up to 60 wt-%, based on the total weight of the compressed tablet. Preferably in an amount of 3-30 wt-%, based on the total weight of the compressed tablet.

The further active ingredients can be added as such and/or as a formulation.

Therefore the present invention relates to a compressed tablet (CT5), which is the compressed tablet (CT), (CT1), (CT1′), (CT2), (CT2′), (CT3), (CT3′), (CT3″), (CT3′″), (CT4), (CT4′), (CT4″) or (CT4′″), wherein the compressed comprises at least one further active ingredient (next to HMOs).

Therefore the present invention relates to a compressed tablet (CT5′), which is the compressed tablet (CT5), wherein the at least one further active ingredient is chosen from the group consisting of vitamins, nutritional lipids, carotenoids, minerals, nutraceuticals, glycosaminoglycan or its active members and amino acids, and any other type of dietary ingredients.

Therefore the present invention relates to a compressed tablet (CT5′), which is the compressed tablet (CT5), wherein the at least one further active ingredient is chosen from the group consisting of vitamins, minerals, glycosaminoglycan and its active members.

Therefore the present invention relates to a compressed tablet (CT5′″), which is the compressed tablet (CT5), (CT5′) or (CT5″), wherein the amount of the at least one further active ingredient is up to 60 wt-%, based on the total weight of the compressed tablet.

Therefore the present invention relates to a compressed tablet (CT5″″), which is the compressed tablet (CT5), (CT5′) or (CT5″), wherein the amount of the at least one further active ingredient is 3-30 wt-%, based on the total weight of the compressed tablet.

The compressed tablet according to the present invention can comprise any commonly known and used auxiliary agents, which are usually used to produce compressed tablets, such as bulking agents, flow aid, sweeteners, flavoring agents, disintegrating agents, preservatives, lubricating agents, and anti-sticking agents. Such ingredients are useful for enhancing the manufacturability, texture and appearance of the product. Those of ordinary skill in the art will be familiar with such inactive ingredients auxiliary agents.

Such additional ingredients may be present in the amount of up to 98 wt-%, based on the total weight of the compressed tablet (preferably 3-60 wt-%, more preferred 5-50 wt-%, based on the total weight of the compressed tablet).

Therefore the present invention relates to a compressed tablet (CT6), which is the compressed tablet (CT), (CT1), (CT1′), (CT2), (CT2′), (CT3), (CT3′), (CT3″), (CT3′″), (CT4), (CT4′), (CT4″), (CT4′″), (CT5), (CT5′), (CT5″), (CT5′″) or (CT5″″), wherein the compressed tablet comprises at least one auxiliary agent.

Therefore the present invention relates to a compressed tablet (CT6′), which is the compressed tablet (CT6), wherein the compressed tablet comprises at least one auxiliary agent chosen from the group consisting of bulking agents, flow aid, sweeteners, flavoring agents, disintegrating agents, preservatives, lubricating agents, and anti-sticking agents.

Therefore the present invention relates to a compressed tablet (CT6″), which is the compressed tablet (CT6) or (CT6′), wherein the amount of the at least one auxiliary agent is up to 98 wt-%, based on the total weight of the compressed tablet.

Therefore the present invention relates to a compressed tablet (CT6′″), which is the compressed tablet (CT6) or (CT6′), wherein the amount of the at least one auxiliary agent is 3-60 wt-%, based on the total weight of the compressed tablet.

Therefore the present invention relates to a compressed tablet (CT6″″), which is the compressed tablet (CT6) or (CT6′), wherein the amount of the at least one auxiliary agent is 5-50 wt-%, based on the total weight of the compressed tablet.

As stated above, it is clear that the wt-% s of a compressertablet always add up to 100.

The size and the shape of the compressed tablet is not essential. It can have any commonly used shape and size. In the context of the present invention the size is defined as the diameter of the longest dimension.

Usually, the compressed tablet has a disc like shape. It can also be in an elliptic form or any desired form.

The size of the compressed tablet is usually so that it can be swallow or chewed easily.

A common size (diameter) for a disc shaped compressed tablet is 3-20 mm. The height of the tablet depends on the tablet weight and compression force, which is applied during its production.

The overall weight of the compressed tablet can also vary. Usually, it is between 0.2 to 5 g.

Furthermore, even not necessary, the compressed tablet according to the present invention can also be coated.

The coated tablets according to the present invention can be stored in any commonly known packages (blisters, container, bags, etc). If wished the compressed tablets could also be packaged individually.

The process for the production of the compressed tablets is done by the use of conventional methods. In the context of the present invention a rotary tablet press is used. Such devices are commercially available from a variety of suppliers. The one which is used for all examples of the present invention is the PICCOLA CLASSIC “B” type rotary tablet press (from SMI).

The hardness of the compressed tablets is between 2 kp to 45 kp (this is measured on a Sotax HT1 hardness tester (from SOTAX)). Preferably, the hardness of the compressed tablets is between 5 kp to 45 kp.

All hardness values in this patent application are measured on a Sotax HT1 hardness tester.

Therefore the present invention relates to a compressed tablet (CT7), which is the compressed tablet (CT), (CT1), (CT1′), (CT2), (CT2′), (CT3), (CT3′), (CT3″), (CT3′″), (CT4), (CT4′), (CT4″), (CT4′″), (CT5), (CT5′), (CT5″), (CT5′″), (CT5″″), (CT6), (CT6′), (CT6″) or (CT6′″), wherein the hardness of the compressed tablets is between 2 kp to 45 kp (this is measured on a Sotax HT1 hardness tester (from SOTAX).

Therefore the present invention relates to a compressed tablet (CT7′), which is the compressed tablet (CT), (CT1), (CT1′), (CT2), (CT2′), (CT3), (CT3′), (CT3″), (CT3′″), (CT4), (CT4′), (CT4″), (CT4′″), (CT5), (CT5′), (CT5″), (CT5′″), (CT5″″), (CT6), (CT6′), (CT6″) or (CT6′″), wherein the hardness of the compresses tablets is between 5 kp to 45 kp (this is measured on a Sotax HT1 hardness tester (from SOTAX).

The following examples serve to illustrate the invention.

### EXAMPLES

### General Procedure for All Examples:

1. Weigh out all ingredients per the batch card.

2. Pass all ingredients (except the premix) through a 20 mesh screen.

3. Add all ingredients except the Magnesium Stearate in a V blender and blend for 10 to 15 mins.

4. Add sieved Magnesium Stearate into step 3 and blend for 2 to 5 mins.

5. Produce tablets on Piccola “B” type rotary press (from RIVA) equipped with 0.5 inch round standard concave toolings. Hardness of tablets was measured on a Sotax HT1 hardness tester.

### Examples 1-12

TABLE 1
Examples 1-5 (Exp. 1-3 are comparative Examples)

| | Exp. 1 | Exp. 2 | Exp. 3 | Exp. 4 | Exp. 5 |
|---|---|---|---|---|---|
| | Amount | Amount | Amount | Amount | Amount |
| Ingredients | [mg] | [mg] | [mg] | [mg] | [mg] |
| HMO LNnT 9000 | | | | 210.0 | |
| HMO 2′FL | | | | | 210.0 |
| Aerosil 200 Pharma | 4.5 | 4.5 | 4.5 | 4.5 | |
| Mannitol | 237.5 | 237.5 | 237.5 | 237.5 | 242.0 |
| Sorbitol | 416.0 | 516.0 | 626.0 | 416.0 | 416.0 |
| Reb-M | 2.0 | 2.0 | 2.0 | 2.0 | 2.0 |
| Citric Acid | 5.0 | 5.0 | 5.0 | 5.0 | 5.0 |
| Malic Acid | 5.0 | 5.0 | 5.0 | 5.0 | 5.0 |
| Orange Flavor | 5.0 | 5.0 | 5.0 | 5.0 | 5.0 |
| Dura-rome | | | | | |
| Nat. Orange Flavor | 6.0 | 6.0 | 6.0 | 6.0 | 6.0 |
| Magnesium Stearate | 9.0 | 9.0 | 9.0 | 9.0 | 9.0 |

TABLE 2
Compression profiles of Examples 1-5

| | Exp. 1 | Exp. 2 | Exp. 3 | Exp. 4 | Exp. 5 |
|---|---|---|---|---|---|
| Compression profiles | (kp) | (kp) | (kp) | (kp) | (kp) |
| 1000 Lbs. | 6.3 | 8.5 | 9.7 | 15.0 | 7.4 |
| 1500 Lbs. | 8.6 | 11.6 | 13.7 | 19.0 | 10.4 |
| 2000 Lbs. | 13.4 | 16.5 | 17.9 | 29.2 | 15.3 |
| 2500 Lbs. | 16.1 | 20.0 | 23.8 | 34.5 | 18.3 |
| 3000 Lbs. | 19.8 | 24.6 | 29.3 | 42.0 | 22.6 |

As can be seen in Table 1, 5 batches of tablets (Examples 1-5) have been produced using the same base formula and the same toolings (0.5″ round standard concave) on the same rotary press (Piccola “B” press). Example 1, 2 and 3 are comparative placebo (with no HMO) batches with different usage rates of sorbitol.

In Table 2, it can be seen tablet hardness increases with the increase of sorbitol in a tablet. Example 4 used 210.0 mg of HMO LNnT per tablet. Example 4 used the same base as Example 1, but the tablet hardness increased more than 2 times than the one of Example 1 when compressed at the same compression force. The tablet hardness of Example 4 also significantly higher than Example 3 which has the same tablet weight as Example 4 and more sorbitol in the formulation (to Q.S). Example 5 comprises HMO 2′FL and used the same base as in Example 1 too. The tablet hardness of Example 5 is higher than the one of Example 1.

TABLE 3
Examples 6-9 (Comparative Examples)

| | Exp. 6 | Exp. 7 | Exp. 8 | Exp. 9 |
|---|---|---|---|---|
| | Amount | Amount | Amount | Amount |
| Ingredients | [mg/tab] | [mg/tab] | [mg/tab] | [mg/tab] |
| HMO LNnT 9000 | | | | |
| HMO 2′FL | | | | |
| Dry Vitamin E | 5.2 | 5.2 | 5.2 | 5.2 |
| 50% CWS/S | | | | |
| d-Biotin 1% | 0.6 | 0.6 | 0.6 | 0.6 |
| Trituration on | | | | |
| Maltodex-trin | | | | |
| Ascorbic Acid | 36.8 | 36.8 | 36.8 | 36.8 |
| Zinc Gluconate | 6.4 | 6.4 | 6.4 | 6.4 |
| OPTISHARP Nat. | 11.1 | 11.1 | 11.1 | 11.1 |
| Zeaxanthin | | | | |
| 5% CWS/S-TG | | | | |
| Melatonin | 1.9 | 1.9 | 1.9 | 1.9 |
| FLORAGLO Lutein | 37.9 | 37.9 | 37.9 | 37.9 |
| 10% CWS/S-TG | | | | |
| Aerosil 200 Pharma | 4.5 | 4.5 | 4.5 | 4.5 |
| Mannitol | 237.5 | 237.5 | 237.5 | 237.5 |
| Sorbitol | 416.0 | 521.0 | 626.0 | 416.0 |
| Microcrystalline | | | | 210.0 |
| Cellulose | | | | |
| Reb-M | 2.0 | 2.0 | 2.0 | 2.0 |
| Citric Acid FG | 5.0 | 5.0 | 5.0 | 5.0 |
| Malic Acid FP | 5.0 | 5.0 | 5.0 | 5.0 |
| Nat. Orange Durarome | 5.0 | 5.0 | 5.0 | 5.0 |
| Nat. Orange Flavor | 6.0 | 6.0 | 6.0 | 6.0 |
| Magnesium Stearate | 9.0 | 9.0 | 9.0 | 9.0 |

TABLE 4
Compression profiles of Examples 6-9

| | Exp. 6 | Exp. 7 | Exp. 8 | Exp. 9 |
|---|---|---|---|---|
| Compression profiles | (kp) | (kp) | (kp) | (kp) |
| 1000 Lbs. | 6.5 | 7.7 | 8.5 | 6.7 |
| 1500 Lbs. | 8.4 | 10.8 | 12.2 | 9.9 |
| 2000 Lbs. | 11.0 | 13.7 | 17.6 | 16.0 |
| 2500 Lbs. | 15.1 | 18.1 | 21.8 | 19.4 |
| 3000 Lbs. | 17.4 | 22.5 | 26.5 | 24.9 |

TABLE 5
Examples 10-12

| | Exp. 10 | Exp. 11 | Exp. 12 |
|---|---|---|---|
| | Amount | Amount | Amount |
| Ingredients | [mg/tab] | [mg/tab] | [mg/tab] |
| HMO LNnT 9000 | 105.0 | 210.0 | |
| HMO 2′FL | | | 210.0 |
| Dry Vitamin E 50% CWS/S | 5.2 | 5.2 | 5.2 |
| d-Biotin 1% Trituration | 0.6 | 0.6 | 0.6 |
| on Maltodextrin | | | |
| Ascorbic Acid | 36.8 | 36.8 | 36.8 |
| Zinc Gluconate | 6.4 | 6.4 | 6.4 |
| OPTISHARP Nat. Zeaxanthin | 11.1 | 11.1 | 11.1 |
| 5% CWS/S-TG | | | |
| Melatonin | 1.9 | 1.9 | 1.9 |
| FLORAGLO Lutein 10% CWS/S-TG | 37.9 | 37.9 | 37.9 |
| Aerosil 200 Pharma | 4.5 | 4.5 | 4.5 |
| Mannitol | 237.5 | 237.5 | 237.5 |
| Sorbitol | 416.0 | 416.0 | 416.0 |
| Microcrystalline Cellulose | | | |
| Reb-M | 2.0 | 2.0 | 2.0 |
| Citric Acid FG | 5.0 | 5.0 | 5.0 |
| Malic Acid FP | 5.0 | 5.0 | 5.0 |
| Nat. Orange Durarome | 5.0 | 5.0 | 5.0 |
| Nat. Orange Flavor | 6.0 | 6.0 | 6.0 |
| Magnesium Stearate | 9.0 | 9.0 | 9.0 |

TABLE 6
Compression profiles of Examples 10-12

| | mg/tablet | | |
|---|---|---|---|
| | Exp. 10 | Exp. 11 | Exp. 12 |
| Compression profiles | (kp) | (kp) | (kp) |
| 1000 Lbs. | 8.1 | 13.1 | 6.7 |
| 1500 Lbs. | 14.0 | 16.2 | 10.6 |
| 2000 Lbs. | 17.8 | 28.5 | 13.1 |
| 2500 Lbs. | 23.1 | 34.5 | 17.6 |
| 3000 Lbs. | 28.8 | 40.1 | 22.5 |

As can be seen in Tables 3-6, 7 batches (examples 6-12) of tablets have been produced using the same base formula and the same toolings (0.5″ round standard concave) on the same rotary press (Piccola “B” press). Different from examples in Tables 1 and 2, all 7 examples have multivitamins, mineral, carotenoids, and other dietary ingredient (Melatonin in this case) in the formulations. Examples 6-9 have no HMO in the formulations (Comparative Examples). Example 7 and 8 have more sorbitol (mg/tab) than Example 6 in the formulation. Example 9 has 210.0 mg/tab more of microcrystalline cellulose than Example 6 and has the same tablet weight with Example 8. Examples 7, 8 and 9 show higher tablet hardness than Example 6 with the increase of binder (sorbitol and/or microcrystalline cellulose). Examples 10, 11 and 12 have the same base formula with Example 6. Example 12 has 210.0 mg/tab of HMO 2′FL in the formulation and shows higher hardness than Example 6. Examples 10 and 11 and have 105.0 mg/tab and 210.0 mg/tab of HMO LNnT in the formulations, respectively. Even with lower amount of HMO LNnT, Example 10 has significantly higher hardness than Example 6. Example 11 has even higher hardness than all other tablet of this series. For example, tablets of Example 11 have a hardness of 40.1 kp when compressed at 3000 Lbs. while tablets of Example 6 only have a hardness of 17.4 kp; and Examples 8 and 9 have hardness of 26.5 kp and 24.9 kp respectively when compressed at 3000 Lbs. As discussed, good binding capacity of HMO helps to achieve good tablet hardness with less amount of binders such as sorbitol and microcrystalline cellulose. In addition, the addition of HMO can significantly reduce the compression force if certain tablet hardness is desired. For example, if we need to achieve around 16 kp tablet hardness, only 1500 Lbs. compression force is needed for Example 11 (with 210.0 mg/tab of HMO LNnT), however the following compression forces are needed: 2500 Lbs. to 3000 Lbs. for Example 6;2000 Lbs. to 2500 Lbs. for Example 7; 2000 Lbs. for Example 8 and 9.

1. A compressed tablet comprising
(i) at least one binding agent
(ii) at least 1 wt-%, based on the total weight of the compressed tablet, of at least one HMO, and
(iii) optionally at least one further active ingredient,
wherein the compressed tablet has a hardness of at least 2 kp.

2. Compressed tablet according to claim 1, wherein the at least binding agent is chosen from the group consisting of saccharides and their derivatives, proteins, synthetic polymers, and mineral type of binders.

3. Compressed tablet according to claim 1, wherein the at least binding agent is chosen from the group consisting of disaccharides, polysaccharides and their derivatives; sugar alcohols; gelatin; polyvinylpyrrolidone (PVP) and polyethylene glycol (PEG) and mineral type of binders.

4. Compressed tablet according to claim 1, wherein the amount of the at least one binding agent is 0.5 to 98 wt-%, based on the total weight of the compressed tablet.

5. Compressed tablet according to claim 1, wherein the HMO are chosen from the group consisting of 2′-fucosyllactose (2′ FL), lacto-N-neotetraose (LNnT), 3-fucosyllactose (3FL), difucosyl-lactose (DFL), Lacto-N-fucopentaose I (LNFP I), 3′Sialyllactose Sodium Salt (3′SL), 6′Sialyllactose Sodium Salt (6′SL), and Lacto-N-Tetraose (LNT).

6. Compressed tablet according to claim 1, wherein the HMO are chosen from the group consisting of 2′-fucosyllactose (2′ FL) and lacto-N-neotetraose (LNnT).

7. Compressed tablet according to claim 1, wherein the content of the at least one HMO is 1-60 wt-%, based on the total weight of the compressed tablet.

8. Compressed tablet according to claim 1, wherein the content of the at least one HMO is 2-55 wt-%, based on the total weight of the compressed tablet.

9. Compressed tablet according to claim 1, wherein the compressed tablet comprises at least one further active ingredient (next to HMOs).

10. Compressed tablet according to claim 9, wherein the at least one further active ingredient is chosen form the group consisting of vitamins, nutritional lipids, carotenoids, minerals, nutraceuticals, glycosaminoglycan or its active members and amino acids, and any other type of dietary ingredient.

11. Compressed tablet according to claim 9, wherein the amount of the at least one further active ingredient is up to 60 wt-%, based on the total weight of the compressed tablet.

12. Compressed tablet according to claim 1, wherein the compressed tablet comprises at least one auxiliary agent.

13. Compressed tablet according to claim 12, wherein the compressed tablet comprises at least one auxiliary agent chosen from the group consisting of bulking agents, sweeteners, flow aid, flavoring agents, disintegrating agents, preservatives, lubricating agents, and anti-sticking agents.
